(19) United States
(12) Patent Application Publication
(10) Pub. No.: US 2009/0081261 A1
(43) Pub. Date: Mar. 26, 2009

(54) Liquid foundation, a makeup method, and a kit for implementing such a method

(75) Inventor: Ludovic Thevenet, Bourg La Reine (FR)

Correspondence Address: OLIFF & BERRIDGE, PLC, P.O. BOX 320850, ALEXANDRIA, VA, 22320-4850, US

(73) Assignee: L'Oreal, Paris (FR)

(21) Appl. No.: 11/922,411

(22) PCT Filed: Jul. 4, 2006
(86) PCT No.: PCT/IB2006/052239

§ 371 Date: Jun. 17, 2008

Related U.S. Application Data
(60) Provisional application No. 60/704,425, filed on Aug. 2, 2005.

(30) Foreign Application Priority Data
Jul. 8, 2005 (FR) 05 52121

(51) Int. Cl. A61K 8/19; A61K 8/02; A61Q 1/08

(52) U.S. Cl. 424/401; 424/63

(57) ABSTRACT

The present invention relates to a foundation including at least one coloring agent having non-zero magnetic susceptibility, the foundation presenting in bulk a hue angle h lying in the range 30° to 70° and lightness L* lying in the range 25 to 80, the coloring agent being selected in such a manner that the foundation, under the action of a magnetic field, can present maximum variation in hue angle Δhₘₐₓ that is less than or equal to 5°, and variation in lightness ΔL* that is greater than or equal to 4.

The present invention relates to making up the skin, in particular the skin of the face or the body.

Light/dark type makeup is sometimes sought to illuminate certain regions of the face, for emphasis, and for hiding regions that are darker.

In the prior art, that type of makeup has required at least two compositions of different lightnesses, which complicates application.

There exists a need to further improve the making up of the body and the face, and for example to make it easier to achieve makeup of the light/dark type.

### Foundation

In one of its aspects, the invention provides a foundation, preferably a liquid foundation, including at least one coloring agent having non-zero magnetic susceptibility, the foundation in bulk presenting a hue angle h lying in the range 30° to 70° and lightness L* lying in the range 25 to 80, the coloring agent being selected in such a manner that the foundation, under the action of a magnetic field, can present maximum variation in hue angle Δhₘₐₓ that is less than or equal to 5°, and variation in lightness ΔL* that is greater than or equal to 4.

Such a foundation is advantageously used by being exposed in part to a magnetic field, thereby enabling variations to be created in the lightness of the foundation between regions that are exposed and other regions.

Such variation in lightness can be useful for achieving makeup of the light/dark type, for example.

In addition, the invention makes it easier to obtain gradual shading between the darker and the lighter regions, since the change in lightness under the effect of the magnetic field can be achieved progressively and reversibly without contacting the deposited layer of foundation, and thus without significantly affecting the uniformity of the deposit.

The parameters h and L* define color in the 1976 L*a*b*CIE color space.

Depending on the complexion for which the composition is intended, the hue angle h of the foundation may lie in the range 35° to 65°, in the range 35° to 55°, or in the range 40° to 60°.

For example, the hue angle h may lie in the range 460 to 500 and the lightness L* may lie in the range 55 to 65, or the hue angle h may lie in the range 49° to 54° and the lightness L* in the range 55 to 65, or the hue angle h may lie in the range 50° to 55° and the lightness L* in the range 60 to 65, or the hue angle h may lie in the range 50° to 55° and the lightness in the range 40 to 55.

The variation in lightness ΔL* may be greater than or equal to 5, or even greater than or equal to 6, thus making it possible to obtain greater contrast between regions exposed to the magnetic field and those that are not exposed thereto.

The viscosity of the foundation advantageously lies in the range 6.5 poise (P) to 38 P, better in the range 11.5 P to 19 P. Such viscosity can encourage retention of the magnetic pigments contained in the coloring agent in the orientation that has been imparted thereto by exposure to the magnetic field. Viscosity is measured at 25° C., using the Rheomat rheometer from the supplier Rheometric Scientific with moving member No. 3 after a duration of 10 minutes (min).

The foundation advantageously includes at least one volatile solvent.

By way of example the volatile solvent may be a volatile oil.

The foundation may also include at least one film-forming polymer.

In another of its aspects, the invention provides a foundation comprising:
at least one aspherical composite pigment comprising a core having non-zero magnetic susceptibility and coated at least in part by a mixture of coloring materials selected so that the foundation presents in bulk lightness L* lying in the range 25 to 80 and a hue angle h lying in the range 30° to 70°.

The core may present an elongate shape, for example.

The core may comprise metallic iron, in particular soft iron.

In another of its aspects, the invention also provides a foundation comprising:
a mixture of at least two aspherical composite pigments each comprising a core having non-zero magnetic susceptibility and coated at least in part in at least one coloring material, the coloring materials of the mixture being selected so that the foundation in bulk presents lightness L* lying in the range 25 to 80, and a hue angle h lying in the range 30° to 70°. The core may comprise metallic iron, in particular soft iron.

In another of its aspects, the invention also provides a method of making up the skin, in particular the face, the method comprising:
depositing a foundation as defined above on the skin, e.g. the skin of the face; and
subjecting all or part of the foundation as deposited in this way on the skin to a magnetic field so as to modify the orientation of all or some of the particles of coloring agent(s) having non-zero magnetic susceptibility.

When the foundation is suitable for drying, in particular because of the presence of one or more volatile compounds, exposure of the deposited foundation to the magnetic field should take place before the composition dries, so that the particles with magnetic susceptibility can move in response to the magnetic field. In addition, exposure to the magnetic field advantageously continues until the particles with magnetic susceptibility have become set within the deposited foundation, such that the visual effect obtained in response to the deposited foundation being exposed to the magnetic field persists, even after the magnetic field has been interrupted.

The modification to the orientation of the coloring agent particles in the foundation is preferably suitable for producing variation in the lightness ΔL* of the foundation deposited on the skin that is greater than or equal to 4, or even greater than or equal to 5 or 6.

This variation in lightness can occur without the hue angle varying by more than 5°, for example.

In an implementation of the method, light/dark type makeup is achieved.

The invention also provides a kit for implementing such a method, the kit comprising:
a receptacle or a backing carrying a foundation as defined above; and
at least one magnetic device enabling a magnetic field to be generated for the purpose of modifying the lightness of the foundation after it has been applied.

The kit may comprise a foundation applicator, e.g. a brush, a foam, a flocked sponge, or woven or non-woven fabric.

By way of example, the backing may be adapted to enable foundation to be transferred onto the skin by being pressed thereagainst. The backing may be preimpregnated with the foundation. Where appropriate, the backing may be insoluble in a solvent that is used during application, such as water, for example.

The receptacle containing the foundation may also be adapted to application without contact, by airbrush, or by spraying by mechanically ejecting droplets, e.g. by means of a vibrator or a print head.

The magnetic device may comprise at least one permanent magnet or at least one electromagnet.

The magnetic device may be distinct from the applicator.

### Magnetic Devices

The magnetic device may comprise a permanent magnet or an electromagnet, e.g. powered by at least one optionally rechargeable battery. With an electromagnet, the magnetic device may include a switch enabling the electromagnet to be electrically powered selectively.

The magnetic device may be arranged to create a magnetic field of orientation that varies over time. When the magnetic device includes a permanent magnet, the device may also include a motor serving to rotate the magnet. In a variant, the magnetic device may comprise a plurality of solenoids disposed in such a manner as to generate a rotating magnetic field when powered sequentially with electricity.

A rotating magnetic field can serve, for example, to obtain a motif that presents circular symmetry, e.g. a motif giving the impression of a sphere in relief.

The electromagnet(s) may be powered continuously or intermittently, as selected by the user. In particular, the magnetic device may be arranged in such a manner that the electromagnet(s) cannot be powered until the magnetic device has been properly positioned close to a surface coated in the first composition.

The magnetic field may be at least 50 milliteslas (mT), or indeed at 0.2 teslas (T), or at least 1 T (10,000 gauss (G)).

To make application of the magnetic field easier, the magnetic device may include a member enabling it to be positioned relative to the surface on which the composition has been deposited. This can serve for example to prevent the magnetic device accidentally coming into contact with the composition, and/or to center the motif produced on the region concerned.

In an embodiment of the invention the magnetic device is secured to an applicator that also serves for applying the cosmetic composition. This can serve to reduce the number of articles handled by the user and can facilitate making up.

In another embodiment of the invention, the magnetic device comprises a permanent magnet mounted at a first end of a stem whose second end is connected to a handle member of an applicator for use in applying the cosmetic composition.

The magnetic field may also be exerted by means of a magnetic structure, in particular a flexible structure, having alternating north and south poles. Such a structure can be used, for example, to produce repetitive motifs on the first composition, e.g. streaks.

The invention also provides a method of promoting the sale of a foundation presenting non-zero magnetic susceptibility, in which changing appearance of the foundation under the effect of a magnetic field is demonstrated. This demonstration may be carried out at a point of sale, for example.

Measuring L* and h° of the Foundation

The values L* and h° characterizing the color of the foundation are measured in bulk as follows. The foundation is placed in a receptacle presenting at least one open surface revealing a disk having a diameter of not less than 8 millimeters (mm) so as to cover completely the aperture of a CM3700d spectrophotometer from the supplier Minolta, used in excluded specular mode.

Measuring the Maximum Variation in Hue Angle Δhₘₐₓ

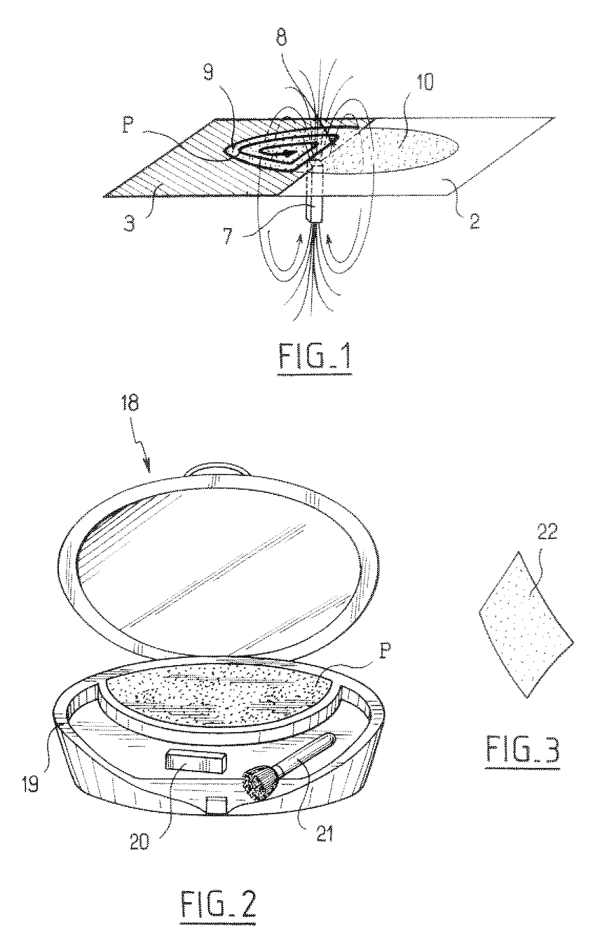

The foundation whose Δhₘₐₓ is to be measured is applied with an automatic spreader with a controlled and uniform thickness of 40 micrometers (μm) on a Gardner trademark contrast card having a thickness of 30 μm, that presents a pale portion 2 and a dark portion 3 as shown in FIG. 1. The apparatus includes a carriage for moving a spreader calibrated to the thickness desired for the deposit that is to be made, together with a suction system enabling the contrast card to be pressed against a plane surface. The foundation is initially deposited prior to spreading so as to cover a rectangle of about 7.5 centimeters (cm) by about 1.3 cm.

L* and h are measured using a Minolta CM3720d spectrophotometer under excluded specular D65 lighting conditions of d/8° configuration.

After spreading, the foundation P covers the black and white backgrounds of the card in part, as can be seen in FIG. 1.

The hue angle and lightness values for the spread foundation are measured before applying any magnetic excitation. These values are written hᵢₙᵢₜᵢₐₗ and L*ᵢₙᵢₜᵢₐₗ and apply to the portion 9 of the deposit on the black background. This portion 9 is then subjected from beneath to magnetic excitation by means of a permanent magnet 7 so that the change in lightness associated with the magnetic field takes place only in the portion 9.

The magnet 7 is oriented in such a manner that its magnetic field lines are substantially perpendicular to the contrast card 1, with the magnet being moved against the bottom face of the contrast card 1 along a path 8 that is generally spiral-wound. The magnet used develops a magnetic field of about 2000 G.

L* and h° are then measured on the portion 9 while exposed in this way to the magnetic field. These values are written L*_{after excitation} and h_{after excitation}.

Δhₘₐₓ is given by |hᵢₙᵢₜᵢₐₗ−h_{after excitation}|

ΔL* is given by |L*ᵢₙᵢₜᵢₐₗ−L*_{after excitation}|

The values for L* and h° on the portion 10 of the spread foundation covering the white background that has not been subjected to magnetic excitation are also measured for verification purposes.

### Coloring Agent

The coloring agent contained in the foundation comprises one or more pigments and/or colorants presenting overall magnetic susceptibility that is not zero, associated with the presence within the coloring agent of at least one magnetic body.

The coloring agent may comprise at least one pigment presenting non-zero magnetic susceptibility, said pigment possibly being the only pigment in the foundation or possibly being mixed with other pigments that present zero or non-zero magnetic susceptibility.

The coloring agent may present a hue angle, in the absence of magnetic excitation, or on the contrary after magnetic excitation, of a value that is advantageously selected as a function of the color of the user's skin, which may for example be Caucasian, Hispanic, Asiatic, or Black, so as to approximate to the real color of the skin.

The coloring agent may be arranged to produce color by an absorption phenomenon. When color is produced by an absorption phenomenon, the coloring agent may comprise at least one magnetic pigment having one or more coloring materials that absorb at least a portion of the visible spectrum.

The coloring agent preferably comprises at least one aspherical magnetic pigment presenting a shape that is elongate. Thus, when the particles of the pigment are subjected to the magnetic field, they tend to become oriented with their long axes in alignment with the field lines, thereby being subjected to a change in orientation which leads to a change in the appearance of the foundation. In addition, during application by spreading, the aspherical shape encourages the particles to take up an orientation that is substantially parallel to the surface on which the deposit is applied. When the magnetic pigment is substantially spherical, its appearance is preferably non-uniform so that a change in orientation leads to a change in appearance.

The coloring agent may include at least one magnetic material selected from nickel, cobalt, iron, and alloys and oxides thereof, in particular Fe₃O₄, and also gadolinium, terbium, dysprosium, erbium, Cu₂MnAl, MnBi, and alloys and oxides thereof. The magnetic material may be of the “soft” or of the “hard” type.

The quantity of magnetic material in the coloring agent is sufficient to enable the appearance of the foundation to depend on exposure to a magnetic field.

The concentration of magnetic bodies may, for example, lie in the range about 0.05% to about 97% by weight, in particular in the range about 0.1% to about 95% by weight, better in the range about 0.1% to about 90% by weight, for example being about 3% by weight.

The size of the magnetic bodies may, for example, lie in the range 1 nanometer (nm) to 700 μm. The term “size” is used to designate the dimension given by the half population statistical grain size distribution known as D50.

The coloring agent may thus comprise at least one magnetic pigment selected from nacres including iron oxide Fe₃O₄. The coloring agent may include at least one magnetic pigment selected from those sold under the trade names: COLORONA BLACKSTAR BLUE, COLORONA BLACKSTAR GREEN, COLORONA BLACKSTAR GOLD, COLORONA BLACKSTAR RED, CLOISONNE NU ANTIQUE SUPER GREEN, MICRONA MATTE BLACK (17437), MICA BLACK (17260), COLORONA PATINA SILVER (17289), and COLORONA PATINA GOLD (117288) from the supplier MERCK, or indeed FLAMENCO TWILIGHT RED, FLAMENCO TWILIGHT GREEN, FLAMENCO TWILIGHT GOLD, FLAMENCO TWILIGHT BLUE, TIMICA NU ANTIQUE SILVER 110 AB, TIMICA NU ANTIQUE GOLD 212 GB, TIMICA NU-ANTIQUE COPPER 340 AB, TIMICA NU ANTIQUE BRONZE 240 AB, CLOISONNE NU ANTIQUE GREEN 828 CB, CLOISONNE NU ANTIQUE BLUE 626 CB, GEMTONE MOONSTONE G 004, CLOISONNE NU ANTIQUE RED 424 CB, CHROMA-LITE BLACK (4498), CLOISONNE NU ANTIQUE ROUGE FLAMBE (code 440 XB), CLOISONNE NU ANTIQUE BRONZE (240 XB), CLOISONNE NU ANTIQUE GOLD (222 CB), and CLOISONNE NU ANTIQUE COPPER (340 XB) from the supplier ENGELHARD.

The color may also be produced at least in part by an interference phenomenon, and the coloring agent may comprise at least one pigment comprising a succession of layers having high and low refractive indices, selected in such a manner as to obtain the desired reflectance spectrum.

### Composite Pigment

Advantageously, the coloring agent comprises at least one composite pigment, comprising a core and a shell surrounding the core at least in part, the shell possibly comprising a single coloring material or a mixture of different pigments or other coloring materials in proportions enabling the desired color to be obtained. The shell may also comprise a succession of layers having different refractive indices, so as to produce a color by an inference phenomenon, as mentioned above.

The core may be inorganic, presenting non-zero magnetic susceptibility and advantageously of aspherical shape, which can enable a change in the orientation of the core to be obtained under the action of a magnetic field, the core tending, for example, to become oriented along the magnetic field lines. When such a composite pigment is subjected to a change in its orientation under the effect of a magnetic field, a relatively large variation in lightness is likely to be obtained, with this variation in lightness being capable of arising without too great a change in hue angle.

The core may comprise at least one magnetic material selected from nickel, cobalt, iron, and alloys and oxides thereof, in particular Fe₃O₄, and also gadolinium, terbium, dysprosium, erbium, CuMmAl, MnBi, and alloys and oxides thereof. The magnetic material may be of the “soft” or of the “hard” type. The core may comprise metallic iron, in particular soft iron.

The core may present a structure that is monolithic or hydrid, e.g. comprising a dispersion of magnetic materials in a synthetic matrix, in particular a polymer or inorganic matrix, e.g. a thermoplastic material.

The core may present a mean size lying in the range about 1 nm to about 100 nm, for example. The term “mean size” is used to designate the dimension given by the half population statistical grain size distribution known as D50. The mean size may be a number mean size determined by analyzing an image (from an electron microscope).

It is also possible to use cores of greater size.

The shell may be non-magnetic, being arranged to produce a color by an absorption phenomenon in at least a portion of the visible spectrum. In a variant, the composite pigment may have a shell that is arranged to produce color by an interference phenomenon.

The shell may include optionally magnetic inorganic pigments, such as, for example, and in addition to those mentioned above and containing Fe₃O₄, CI74160 Prussian blue, or indeed iron.

The shell may also include at least one organic coloring material, for example at least one organic pigment, in particular at least one organic lake or other organic coloring materials, which can be selected from the following components and mixtures thereof:
cochineal red;
organic pigments of azo, anthraquinone, indigo, xanthene, pyrene, quinoline dyes, triphenylmethane, and fluorane;
organic lakes or insoluble salts of sodium, potassium, calcium, barium, aluminum, zirconium, strontium, titanium, acid dyes such as azo, anthraquinone, indigo, xanthene, pyrene, quinoline dyes, triphenylmethane and fluorane dyes, said dyes possibly comprising at least one carboxylic or sulfonic acid group.

Organic pigments which may be mentioned are those known by the following denominations: D&C Blue No 4, D&C Brown No 1, D&C Green No 5, D&C Green No 6, D&C Orange No 4, D&C Orange No 5, D&C Orange No 10, D&C Orange No 11, D&C Red No 6, D&C Red No 7, D&C Red No 17, D&C Red No 21, D&C Red No 22, D&C Red No 27, D&C Red No 28, D&C Red No 30, D&C Red No 31, D&C Red No 33, D&C Red No 34, D&C Red No 36, D&C Violet No 2, D&C Yellow No 7, D&C Yellow No 8, D&C Yellow No 10, D&C Yellow No 11, FD&C Blue No 1, FD&C Green No 3, FD&C Red No 40, FD&C Yellow No 5, FD&C Yellow No 6.

The organic dye may comprise an organic lake supported by an organic support such as colophane or aluminum benzoate, for example.

Particular examples of organic lakes which may be mentioned are those known by the following denominations: D&C Red No 2 Aluminum lake, D&C Red No 3 Aluminum lake, D&C Red No 4 Aluminum lake, D&C Red No 6 Aluminum lake, D&C Red No 6 Barium lake, D&C Red No 6 Barium/Strontium lake, D&C Red No 6 Strontium lake, D&C Red No 6 Potassium lake, D&C Red No 7 Aluminum lake, D&C Red No 7 Barium lake, D&C Red No 7 Calcium lake, D&C Red No 7 Calcium/Strontium lake, D&C Red No 7 Zirconium lake, D&C Red No 8 Sodium lake, D&C Red No 9 Aluminum lake, D&C Red No 9 Barium lake, D&C Red No 9 Barium/Strontium lake, D&C Red No 9 Zirconium lake, D&C Red No 10 Sodium lake, D&C Red No 19 Aluminum lake, D&C Red No 19 Barium lake, D&C Red No 19 Zirconium lake, D&C Red No 21 Aluminum lake, D&C Red No 21 Zirconium lake, D&C Red No 22 Aluminum lake, D&C Red No 27 Aluminum lake, D&C Red No 27 Aluminum/Titanium/Zirconium lake, D&C Red No 27 Barium lake, D&C Red No 27 Calcium lake, D&C Red No 27 Zirconium lake, D&C Red No 28 Aluminum lake, D&C Red No 30 lake, D&C Red No 31 Calcium lake, D&C Red No 33 Aluminum lake, D&C Red No 34 Calcium lake, D&C Red No 36 lake, D&C Red No 40 Aluminum lake, D&C Blue No 1 Aluminum lake, D&C Green No 3 Aluminum lake, D&C Orange No 4 Aluminum lake, D&C Orange No 5 Aluminum lake, D&C Orange No 5 Zirconium lake, D&C Orange No 10 Aluminum lake, D&C Orange No 17 Barium lake, D&C Yellow No 5 Aluminum lake, D&C Yellow No 5 Zirconium lake, D&C Yellow No 6 Aluminum lake, D&C Yellow No 7 Zirconium lake, D&C Yellow No 10 Aluminum lake, FD&C Blue No 1 Aluminum lake, FD&C Red No 4 Aluminum lake, FD&C Red No 40 Aluminum lake, FD&C Yellow No 5 Aluminum lake, FD&C Yellow No 6 Aluminum lake.

The chemical compounds corresponding to each of the organic dyes mentioned above are mentioned in the work “International Cosmetic Ingredient Dictionary and Handbook”, 1997 edition, pages 371 to 386 and 524 to 528, published by “The Cosmetic, Toiletry and Fragrance Association” the contents of which are hereby incorporated by reference.

The composite pigment may include a binder enabling the coloring shell to adhere to the surface of the core.

### Binder

The binder may be selected in particular from a non-limiting list comprising silicone polymeric compounds, polymeric, oligomeric, or similar compounds, in particular selected from organosilanes, fluoroalkyl organosilanes, and polysiloxanes, e.g. polymethyl hydrogen siloxane, and also various coupling agents such as coupling agents based on silanes, titanates, aluminates, zirconates, and mixtures thereof.

The silicone compound may be selected from a non-limiting list comprising in particular:
organosilanes (1) obtained from alkoxysilanes;
optionally modified polysiloxanes (2) selected from a non-limiting list comprising:
   modified polysiloxanes (2A) including at least one radical selected from, in particular: polyethers, polyesters, and epoxy compounds (referred to “modified polysiloxanes”); and
   polysiloxanes (2B) having a silicon atom situated at the end of the polymer carrying at least one group selected from a non-limiting list comprising carboxylic acids, alcohols and hydroxy groups; and
fluoroalkyl organosilanes (3) obtained from fluoroalkylsilanes.

The organosilane compounds (I) may be obtained from alkoxysilane compounds presented by the formula (I):

R¹ₐSiX₄₋ₐ (I)

in which:
R¹ represents C₆H₅—, (CH₃)₂CH—CH₂—, or a radical of the C_{b}H_{2b+1}-type (where b lies in the range 1 to 18);
X represents CH₃O— or C₂H₅O—, and
a lies in the range 0 to 3.

Specific examples of alkoxysilane compounds may include the alkoxysilanes selected from: methyltriethoxysilane, dimethyldiethoxysilane, phenyltriethyoxysilane, diphenyldiethoxysilane, methyltrimethoxysilane, dimethyldimethoxysilane, phenyltrimethoxysilane, diphenyldimethoxysilane, isobutyltrimethoxysilane, decyltrimethoxysilane and the like, in particular from methyltriethoxysilane, phenyltriethoxysilane, methyltrimethoxysilane, dimethyldimethoxysilane, sobutyltrimethoxysilane, or better still methyltriethoxysilane, methyltrimethoxysilane, and phenyltriethoxysilane.

The polysiloxanes (2) may satisfy in particular formula (II):

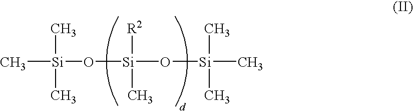

where R² represents H— or CH₃—, and where d lies in the range 15 to 450.

Among these polysiloxanes, those to which R² represents H are preferred.

The modified polysiloxanes (2A) may satisfy in particular the following formula:
(a¹) modified polysiloxanes carrying polyethers, represented by formula (III):

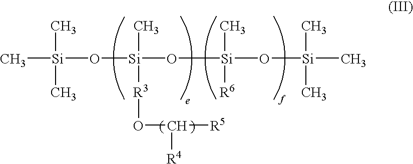

in which R³ represents —(CH₂)ₕ—; R⁴ represents —(CH₂)ᵢ—CH₃; R⁵ represents —OH, —COOH, —CH═CH₂, —C(CH₃)═CH₂ or —(CH₂)ⱼ—CH₃; R⁶ represents —(CH₂)ₖ—CH₃; with g and h lying independently in the range 1 to 15; j and k lying independently in the range 1 to 15; e lying in the range 1 to 50, and f lying in the range 1 to 300;
(a²) modified polysiloxanes carrying polyesters, represented by formula (IV):

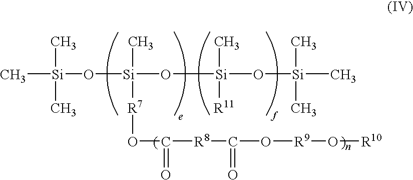

in which R⁷, R⁸, and R⁹ represent independently —(CH₂)_{q}—; R¹⁰ represents —OH; —COOH, —CH═CH₂, —C(CH₃)═CH₂, or —(CH₂)ᵣ—CH₃; R¹¹ represents —(CH₂)ₛ—CH₃; n and q lying independently in the range 1 to 15, r and s lying independently in the range 0 to 15; e lying in the range 1 to 50; and f lying in the range 1 to 300;
(a³) modified polysiloxanes carrying epoxy radicals represented by formula (V):

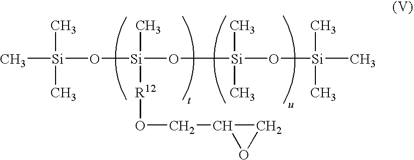

in which R¹² represents —(CH₂)ᵥ—; v lying in the range 1 to 15; t lying in the range 1 to 50; and u lying in the range 1 to 300; or mixtures thereof.

Amongst modified polysiloxanes (2A), modified polysiloxanes carrying polyethers of formula (III) are preferred.

The modified polysiloxanes on the terminal portion (2B) may satisfy formula (VI):

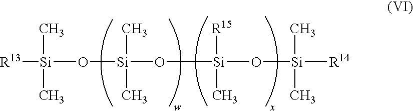

in which R¹³ and R¹⁴ may represent —OH, R¹⁶ may represent —OH, or R¹⁷ may represent —COOH, independently of one another; R¹⁵ represents —CH₃ or —C₆H₅; R¹⁶ and R¹⁷ represent —(CH₂)_{y}—; Y lying in the range 1 to 15; w lying in the range 1 to 200; and x lying in the range 0 to 100.

Amongst these polysiloxanes modified at least one end, those carrying at least one (R¹⁶ and/or R¹⁷) radical carrying a carboxylic acid group on at least one terminal silicon acid are more preferred.

The fluoroalkyle organosiliane compounds (3) may be obtained from fluoroalkyle silanes represented by formula (VII):

CF₃ (CF₂)_{z}CH₂CH₂(R¹⁸)ₐSiX₄₋ₐ (VII)

in which:
R¹⁸ represents CH₃—, C₂H₅—, CH₃O—, or C₂H₅O—;
X represents CH₃O— or C₂H₅O—;
Z lies in the range 0 to 15, and a lies in the range 0 to 3.

The fluoroalkylsilanes may be selected in particular from a non-limiting list comprising in particular: trifluoropropyltrimethoxysilane, tridecafluorooctyltrimethoxysilane, heptadecafluorodecyltrimethoxysilane, heptadecafluorodecylmethyldimethoxysilane, trifluoropropyltriethoxysilane, tridecafluorooctyltriethoxysilane, heptadecafluorodecyltriethoxysilane, heptadecafluorodecylmethyldiethoxysilane and the like, in particular trifluoropropyltrimethoxysilane, tridecafluorooctyltrimethoxysilane, and heptadecafluorodecyltrimethoxysilane, and even better trifluoropropyl trimethoxysilane, and tridecafluorooctyltrimethoxysilane.

The silane-based coupling agents may be selected from the non-limiting list comprising in particular: vinyltrimethoxysilane, vinyltriethoxysilane, γ-aminopropyl-triethoxysilane, γ-glycidoxypropyltrimethoxysilane, γ-aminopropyltriethoxysilane, γ-glycidoxypropyltrimethoxysilane, γ-mercaptopropyltrimethoxysilane, γ-methacryloxypropyltrimethoxysilane, N-β(aminoethyl)-γ-aminopropyltrimethoxysilane, γ-glycidoxypropylmethyldimethoxysilane, γ-chloropropyltrimethoxysilane, and the like.

The titanate-based coupling agents may be selected from the list comprising isopropylstearoyle titanate, isopropyltris(dioctylpyrophosphate) titanate, isopropyl-tri(N-aminoethyl-aminoethyl) titanate, tetraoctyl-bis(ditridecylphosphate) titanate, tetra(2,2-diaryloxymethyl-1-butyl)bis(ditridecyl)phosphate titanate, bis(dioctylpyrophosphate)oxyacetate titanate, bis(dioctylpyrophosphate)ethylene titanate, and the like.

The aluminate-based coupling agents may be selected from acetoalkoxyaluminum diisopropylate, aluminum diisopropoxymonoethylacetoacetate, aluminum triethylacetoacetate, aluminum triacetylacetonate, and the like.

The zirconate-based coupling agents may be selected from the list comprising in particular: zirconium tetrakisacetylacetonate, zirconium dibutoxybisacetylacetonate zirconium tetrakisethylacetoacetate, zirconium tributoxymonoethylacetoacetate, zirconium tributoxyacetylacetonate, and the like.

The compounds acting as a binder may in particular present a molar mass lying in the range 300 to 100,000.

### Preparing the Composite Pigment

The composite pigment presenting a magnetic core and a coloring shell may be prepared by any appropriate method, for example a mechano-chemical method or a method of precipitation in solution, with dissolution of a coloring material followed by precipitation onto the surface of the core.

A binder may optionally be used.

It is possible to use a method involving mechanically mixing one or more pigments and the core.

The binder may be added and mixed with the core before introducing the coloring material.

The composite pigment may be made, for example, using one of the methods described in European patent applications Nos. EP 1 184 426 and EP 1 217 046, the contents of which are incorporated herein by reference.

In one implementation, the first step is to mix the particles that are to constitute the core with the binder.

In order to ensure that the binder adheres uniformly to the surface of the core, it is preferable to begin by passing the particles through a grinder so as to break up any clumps.

The mixing and stirring conditions are selected so that the core is covered uniformly in binder. These conditions can be controlled so that the linear load lies in the range 19.6 newtons per centimeter (N/cm) to 19,160 N/cm, and in particular in the range 98 N/cm to 14,170 N/cm, and better in the range 147 N/cm to 980 N/cm; the processing time lies in particular in the range 5 min to 24 hours (h) and better in the range 10 min to 20 h; the speed of rotation may lie in the range 2 revolutions per minute (rpm) to 1000 rpm, and in particular in the range 5 rpm to 1000 rpm, and better in the range 10 rpm to 800 rpm.

After the binder has covered the core, the material that is to form the shell is added and mixed in with stirring in order to adhere to the binder layer.

The methods of addition may, for example, be addition in large quantity, continuous, or in full quantity.

The mixing and stirring, whether of the cores with the binder or of the material that is to form the shell, can be undertaken using an apparatus suitable for applying a spatula cutting force and/or compression force to the powder mixture. Such apparatuses are constituted, for example, by wheel, blade, and similar mixers or mills. Wheel mills are particularly suitable. A list of suitable apparatuses can be found in application EP 1 184 426 A2.

Another method of fabricating a composite pigment is described in patent JP 3 286 463, which discloses a method of precipitation in solution.

The coloring material is dissolved in ethanol, the cores are then dispersed in the ethanol solution. Thereafter, an alkaline aqueous solution of sodium or potassium carbonate is added slowly to the mixtures, and finally an ethanol solution of calcium chloride is added slowly, stirring throughout.

### Coloring Agents and Pigments

In addition to one or more magnetic pigments, the coloring agent may also include at least one non-metallic pigment or colorant, in order to create a background color independently of the application of a magnetic field. The background color may present a color that is close to the complexion of the person on whom the composition is to be applied.

The dyes may be liposoluble or hydrosoluble.

Examples of liposoluble dyes are Sudan red, DC Red 17, DC Green 6, β-carotene, soya oil, Sudan brown, DC Yellow 11, DC Violet 2, DC orange 5, quinoline yellow.

Examples of hydrosoluble dyes are beetroot juice and methylene blue.

As an example, the dyes may represent 0.1% to 20% of the weight of composition P, or even 0.1% to 6%, when present.

The organic lakes or pigments may be selected from the materials below and their mixtures:
cochineal red;
organic pigments of azo, anthraquinone, indigo, xanthene, pyrene, quinoline dyes, triphenylmethane, and fluorane;
organic lakes or insoluble salts of sodium, potassium, calcium, barium, aluminum, zirconium, strontium, titanium, acid dyes such as azo, anthraquinone, indigo, xanthene, pyrene, quinoline dyes, triphenylmethane and fluorane dyes, said dyes possibly comprising at least one carboxylic or sulfonic acid group.

Organic pigments which may be mentioned are those known by the following denominations: D&C Blue No 4, D&C Brown No 1, D&C Green No 5, D&C Green No 6, D&C Orange No 4, D&C Orange No 5, D&C Orange No 10, D&C Orange No 11, D&C Red No 6, D&C Red No 7, D&C Red No 17, D&C Red No 21, D&C Red No 22, D&C Red No 27, D&C Red No 28, D&C Red No 30, D&C Red No 31, D&C Red No 33, D&C Red No 34, D&C Red No 36, D&C Violet No 2, D&C Yellow No 7, D&C Yellow No 8, D&C Yellow No 10, D&C Yellow No 11, FD&C Blue No 1, FD&C Green No 3, FD&C Red No 40, FD&C Yellow No 5, FD&C Yellow No 6.

The organic dye may comprise an organic lake supported by an organic support such as colophane or aluminum benzoate, for example.

Particular examples of organic lakes which may be mentioned are those known by the following denominations: D&C Red No 2 Aluminum lake, D&C Red No 3 Aluminum lake, D&C Red No 4 Aluminum lake, D&C Red No 6 Aluminum lake, D&C Red No 6 Barium lake, D&C Red No 6 Barium/Strontium lake, D&C Red No 6 Strontium lake, D&C Red No 6 Potassium lake, D&C Red No 7 Aluminum lake, D&C Red No 7 Barium lake, D&C Red No 7 Calcium lake, D&C Red No 7 Calcium/Strontium lake, D&C Red No 7 Zirconium lake, D&C Red No 8 Sodium lake, D&C Red No 9 Aluminum lake, D&C Red No 9 Barium lake, D&C Red No 9 Barium/Strontium lake, D&C Red No 9 Zirconium lake, D&C Red No 10 Sodium lake, D&C Red No 19 Aluminum lake, D&C Red No 19 Barium lake, D&C Red No 19 Zirconium lake, D&C Red No 21 Aluminum lake, D&C Red No 21 Zirconium lake, D&C Red No 22 Aluminum lake, D&C Red No 27 Aluminum lake, D&C Red No 27 Aluminum/Titanium/Zirconium lake, D&C Red No 27 Barium lake, D&C Red No 27 Calcium lake, D&C Red No 27 Zirconium lake, D&C Red No 28 Aluminum lake, D&C Red No 30 lake, D&C Red No 31 Calcium lake, D&C Red No 33 Aluminum lake, D&C Red No 34 Calcium lake, D&C Red No 36 lake, D&C Red No 40 Aluminum lake, D&C Blue No 1 Aluminum lake, D&C Green No 3 Aluminum lake, D&C Orange No 4 Aluminum lake, D&C Orange No 5 Aluminum lake, D&C Orange No 5 Zirconium lake, D&C Orange No 10 Aluminum lake, D&C Orange No 17 Barium lake, D&C Yellow No 5 Aluminum lake, D&C Yellow No 5 Zirconium lake, D&C Yellow No 6 Aluminum lake, D&C Yellow No 7 Zirconium lake, D&C Yellow No 10 Aluminum lake, FD&C Blue No 1 Aluminum lake, FD&C Red No 4 Aluminum lake, FD&C Red No 40 Aluminum lake, FD&C Yellow No 5 Aluminum lake, FD&C Yellow No 6 Aluminum lake.

The chemical compounds corresponding to each of the organic dyes mentioned above are mentioned in the work “International Cosmetic Ingredient Dictionary and Handbook”, 1997 edition, pages 371 to 386 and 524 to 528, published by “The Cosmetic, Toiletry and Fragrance Association” the contents of which are hereby incorporated by reference.

The foundation may include at least one filler.

### Fillers

The term “filler” means particles of any form, which are insoluble in the medium of the composition, regardless of the temperature at which the composition is manufactured. A filler may in particular act to modify the rheology or texture of the composition. The nature and quantity of particles may depend on the desired mechanical properties and the textures.

Examples of fillers which may be mentioned amongst others are talc, mica, silica, kaolin, sericite, powders of polyamide, polyolefins, for example polyethylene, polytetrafluoroethylene, polymethylmethacrylate, polyurethane, starch powders, and silicone resin beads.

The fillers may serve, amongst other purposes, to create a blurring effect in order to hide imperfections of the skin.

### Other Components

The foundation includes a physiologically acceptable medium that is suitable for application to the skin.

The foundation may include ingredients other than those described above, in particular at least one solvent, a fatty phase, a film-forming polymer, and/or agent that is dermatologically or cosmetically active.

### Solvents

The composition may comprise at least one aqueous or organic solvent, in particular at least one volatile organic solvent, especially a volatile organic oil.

Within the context of the present invention, the term “volatile solvent” means a solvent which is liquid at ambient temperature, having a non-zero vapor pressure, at ambient temperature and atmospheric pressure, in particular having a vapor pressure of 0.13 Pa [Pascals] to 40000 Pa (10⁻³ mm Hg to 300 mm Hg), preferably 1.3 Pa to 13000 Pa (0.01 mm Hg to 100 mm Hg), preferably 1.3 Pa to 1300 Pa (0.01 mm Hg to 10 mm Hg).

When composition P comprises one or more organic solvents, said solvents may be present in an amount of 0.1% to 99% with respect to the total weight of the composition in question.

In general, the quantity of solvent(s), in particular organic solvent, depends on the nature of the surface onto which the composition is intended to be applied.

The composition may comprise at least one volatile solvent constituted by a volatile oil.

The oil may be a silicone oil or a hydrocarbon oil, or it may comprise a mixture of said oils.

In the context of the present invention, the term “silicone oil” means an oil comprising at least one atom of silicon, in particular at least one Si—O group.

The term “hydrocarbon oil” means an oil principally containing atoms of hydrogen and carbon, and possibly also atoms of oxygen, nitrogen, sulfur, and/or phosphorus.

The volatile hydrocarbon oils may be selected from hydrocarbon oils containing 8 to 16 carbon atoms, in particular C₈-C₁₆ branched alkanes (also termed isoparaffins) such as isododecane (also termed 2,2,4,4,6-pentamethylheptane), isodecane, isohexadecane and, for example, oils sold under the trade names ISOPARS® or PERMETHYLS®.

Volatile oils which may also be used are volatile silicones, such as volatile linear or cyclic silicone oils, in particular those with a viscosity≦8 centistokes (8×10⁻⁶ m²/s), especially containing 2 to 10 silicon atoms, in particular 2 to 7 silicon atoms, said silicones optionally comprising alkyl or alkoxy groups containing 1 to 10 carbon atoms. Volatile silicone oils which may be used in the invention which may be mentioned are dimethicones with a viscosity of 5 cSt [centistokes] to 6 cSt, octamethyl cycloetrasiloxane, decamethyl cyclopentasiloxane, dodecamethyl cyclohexasiloxane, heptamethyl hexyltrisiloxane, heptamethyloctyl trisiloxane, hexamethyl disiloxane, octamethyl trisiloxane, decamethyl tetrasiloxane, dodecamethyl pentasiloxane, and mixtures thereof.

Mention may also be made of volatile alkyltrisiloxane linear oils having general formula (I):

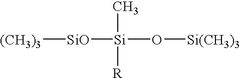

where R represents an alkyl group having 2 to 4 carbon atoms and in which one or more hydrogen atoms can be substituted by a fluorine atom or a chlorine atom.

Amongst oils having general formula (I), mention can be made:
3-butyl 1,1,1,3,5,5,5-heptamethyl trisiloxane,
3-propyl 1,1,1,3,5,5,5-heptamethyl trisiloxane, and
3-ethyl 1,1,1,3,5,5,5-heptamethyl trisiloxane,
   corresponding to oils of formula (I) in which R is respectively a butyl group, a propyl group, or an ethyl group.

It is also possible to use fluorinated volatile oils such as nonafluoromethoxybutane or perfluoromethylcyclopentane, and mixtures thereof.

A composition of the invention may include, for example 0.01% to 95% by weight of volatile oil, relative to the total weight of the composition, and better 1% to 75% by weight.

The composition may comprise at least one organic solvent selected from the following list:
ketones which are liquid at ambient temperature, such as methylethylketone, methylisobutylketone, diisobutylketone, isophorone, cyclohexanone, acetone;
alcohols which are liquid at ambient temperature, such as ethanol, isopropanol, diacetone alcohol, 2-butoxyethanol, cyclohexanol;
glycols which are liquid at ambient temperature, such as ethylene glycol, propylene glycol, pentylene glycol, glycerol;
propylene glycol ethers which are liquid at ambient temperature, such as propylene glycol monomethyl ether, propylene glycol monomethyl ether acetate, dipropylene glycol mono n-butyl ether;
short chain esters (containing 3 to 8 carbon atoms in total), such as ethyl acetate, methyl acetate, propyl acetate, n-butyl acetate, isopentyl acetate;
alkanes which are liquid at ambient temperature, such as decane, heptane, dodecane, cyclohexane.

The composition may also comprise water or a mixture of water and hydrophilic organic solvents routinely used in cosmetics such as alcohols and especially linear or branched lower mono-alcohols containing 2 to 5 carbon atoms, such as ethanol, isopropanol or n-propanol, polyols such as glycerin, diglycerin, propylene glycol, sorbitol, penthylene glycol, polyethylene glycols. Composition P may also contain C₂ ethers and hydrophilic C₂-C₄ aldehydes. The water or mixture of water and hydrophilic organic solvents may be present in the composition in an amount which is, for example, from 0 to 90%, in particular 0.1% to 90% by weight, preferably 0 to 60% by weight, in particular 0.1% to 60% by weight with respect to the total composition weight.

### Fatty Phase

The composition, for example when intended to be applied to the lips, may comprise a fatty phase and in particular at least one fat which is liquid at ambient temperature (25° C.) and/or a fat which is solid at ambient temperature, such as waxes, pasty fats, gums, and mixtures thereof. Further, the fatty phase may contain lipophilic organic solvents.

The composition may, for example, have a continuous fatty phase, which may contain less than 5% water, in particular less than 1% of water with respect to its total weight and in particular it may be in the anhydrous form.

Fats which are liquid at ambient temperature, often termed “oils” which may be mentioned are: vegetable hydrocarbon oils such as liquid triglycerides of fatty acids containing 4 to 10 carbon atoms, such as heptanoic or octanoic acid triglycerides, or sunflower seed, corn, soya, grapeseed, sesame, apricot, macadamia, castor or avocado oils, triglycerides of caprylic/capric acid, jojoba oil, shea butter oil, lanolin, acetylated lanolin, linear or branched hydrocarbons of mineral or synthetic origin such as paraffin oils and derivatives thereof, Vaseline, polydecenes, hydrogenated polyisobutene such as Parleam; synthesized esters and ethers such as those from fatty acids, such as Purcellin oil, isopropyl myristate, 2-ethylhexyl palmitate, 2-octyldodecyl stearate, 2-octyldodecyl erucate, isostearyl isostearate; hydroxylated esters such as isostearyl lactate, octylhydroxystearate, octyldodecyl hydroxystearate, diisostearylmalate, triisocetyl citrate, heptanoates, octanoates, decanoates of fatty alcohols; isononyl isonanoate, isopropyl lanolate, tridecyl trimellilate, diisostearyl malate; polyol esters such as propylene glycol dioctanoate, neopentylglycol diheptanoate, diethylene glycol diisononanoate; pentaeythritol esters; fatty alcohols containing 12 to 26 carbon atoms, such as octyldodecanol, 2-butyloctanol, 2-hexyldecanol, 2-undecylpentadecanol, oleic alcohol; partially fluorinated hydrocarbon and/or silicone oils; silicone oils such as polymethylsiloxanes (PDMS), volatile or otherwise, linear or cyclic, liquid or pasty at ambient temperature such as cyclomethicones, dimethicones, optionally comprising a phenyl group, such as phenyltrimethicones, phenyltrimethylsiloxydiphenylsiloxanes, diphenylmethyldimethyl-trisiloxanes, diphenyldimethicones, phenyl dimethicones, polymethylphenyl siloxanes; and mixtures thereof. The oils may be present in an amount of 0.01% to 90%, preferably 0.1% to 85% by weight with respect to the total composition weight.

The presence of an oily phase may provide gloss and, for example, a refractive index in the range 1.47 to 1.51, preferably in the range 1.48 to 1.50. The refractive index is measured at ambient temperature (25° C.) using a refractometer.

Pasty fats are generally hydrocarbon compounds with a melting point in the range 25° C. to 60° C., preferably in the range 30° C. to 45° C., and/or a hardness in the range 0.001 MPa to 0.5 MPa, preferably in the range 0.005 MPa to 0.4 MPa, such as lanolins and derivatives thereof.

The waxes may be solid at ambient temperature (25° C.), with a reversible solid/liquid change of state, having a melting temperature of more than 30° C. which may be up to 200° C., with a hardness of more than 0.5 MPa, and having an anisotropic crystalline organization in the solid state. In particular, the waxes may have a melting point of more than 25° C., preferably more than 45° C. The waxes may be hydrocarbon, fluorinated, and/or silicone waxes and be of vegetable, mineral, animal, and/or synthetic origin. Waxes which may be used which may be mentioned are beeswax, carnauba wax or candellila wax, paraffin, microcrystalline waxes, ceresine or ozokerite; synthetic waxes such as polyethylene waxes or Fischer-Tropsch waxes, silicone waxes such as alkyl or alkoxy-dimethicone waxes containing 16 to 45 carbon atoms. The composition may contain 0 to 50% by weight of waxes relative to the total composition weight, or even 1% to 30% by weight.

Gums which may be used are generally polydimethylsiloxanes (PDMS) with a high molecular weight, cellulose gums or polysaccharides.

### Film-Forming Polymers

The composition may also, for example, comprise a film-forming polymer, in particular in the case of a mascara or a nail varnish. The term “film-forming polymer” designates a polymer which may, by itself or in the presence of an auxiliary film-forming agent, form a continuous film which adheres to a surface, in particular to keratinous materials.

Examples of film-forming polymers which may be used in composition P which may be mentioned include synthetic polymers, of the radical or polycondensate type, polymers of natural origin such as nitrocellulose or cellulose esters, and mixtures thereof.

Radical type film-forming polymers may be vinyl polymers or copolymers, in particular acrylic polymers.

Vinyl film-forming polymers may result from polymerizing monomers with an ethylenically unsaturated bond containing at least one acid group and/or esters of said acid monomers and/or amides of said acid monomers like unsaturated α,β-ethylenic carboxylic acids such as acrylic acid, methacrylic acid, crotonic acid, maleic acid, or itaconic acid.

Vinyl film-forming polymers may also be the result of homopolymerization or copolymerization of monomers selected from vinyl esters such as vinyl acetate, vinyl neodecanoate, vinyl pivalate, vinyl benzoate or vinyl t-butyl benzoate and styrene monomers such as styrene and alpha-methyl styrene.

Film-forming polycondensates which may be mentioned include polyurethanes, polyesters, polyester amides, polyamides and polyureas, this list being non-limiting.

Polymers of natural origin which may be modified may be selected from shellac resin, sandarac gum, dammar resins, elemi gums, copal gums, cellulose polymers such as nitrocellulose, ethyl cellulose or nitrocellulose esters selected, for example, from cellulose acetate, cellulose acetobutyrate and cellulose acetopropionate, and mixtures thereof.

The film-forming polymer may be present in the form of solid particles in aqueous or oily dispersion, generally known as latexes or pseudolatexes. The film-forming polymer may comprise one or more stable dispersions of particles of generally spherical polymers of one or more polymers in a physiologically acceptable liquid fatty phase. Said dispersions are generally termed NAD (non aqueous dispersions) of polymers as opposed to latexes, which are aqueous dispersions of a polymer. Said dispersions may be in the form of nanoparticles of polymers in stable dispersion in said fatty phase. The nanoparticles preferably have a size in the range 5 nm to 600 nm. Techniques for preparing such dispersions are well known to the skilled person.

Examples of aqueous film-forming polymer dispersions which may be used are acrylic dispersions sold under the trade name NEOCRYL XK-90°, NEOCRYL A-1070®, NEOCRYL A-1090®, NEOCRYL BT-62®, NEOCRYL A-1079®, NEOCRYL A-523® by AVECIA-NEORESINS, DOW LATEX 432® by DOW CHEMICAL, DAITOSOL 5000 AD® by DAITO KASEI KOGYO; or aqueous dispersions of polyurethane sold under the trade name NEOREZ R-981®, NEOREZ R-9740 by AVECIA-NEORESINS, AVALURE UR-405®, AVALURE UR-410®, AVALURE UR-425®, AVALURE UR-450®, SANCURE 875®, SANCURE 861®, SANCURE 878®, SANCURE 2060® by GOODRICH, IMPRANIL 85® from BAYER, AQUAMERE H-1511 by HYDROMER; sulfopolyesters sold under the trade name Eastman AQ by Eastman Chemical Products.

### Sequenced Film-Forming Polymer

In an embodiment of the invention, the foundation includes at least one film-forming polymer that is an ethylene polymer, in particular a sequenced acrylic polymer.

In the meaning of the invention, the term “sequenced acrylic polymer” is used to mean an acrylic block copolymer and having at least one sequence (or block) of limited conformation and at least one sequence (or block) of variable conformation. These two sequences are incompatible with each other and they are characterized by different glass transition temperatures (Tg).

The first and second sequences may be connected together by an intermediate segment comprising at least one component monomer of the first sequence and at least one component monomer of the second sequence.

In the meaning of the present invention, the terms “block”, “sequence”, or “portion” mean a repetitive chain of monomer units, the repetition being equal to at least 2 units, in particular to at least 3 units, and in particular not less than 5 units, or even at least 7 units. The terms “block”, “sequence”, and “portion” are used as equivalents.

In the meaning of the present invention, the monomers are organized in such a manner that the sequenced polymer presents at least one block of variable conformation.

Generally, the block possesses a glass transition temperature that is less than 30° C., in particular lying in the range −100° C. to 25° C., or in the range −90° C. to 20° C., and better in the range −80° C. to 0° C.

The term “glass transition temperature” refers to the temperature at which a polymer goes from the rigid state to a flexible state.

The glass transition temperature of a copolymer can be calculated theoretically using the following formula:

$$\frac{1}{Tg} = \frac{W 1}{Tg 1} + \frac{W 2}{Tg 2} + \frac{W 3}{Tg 3} + … + \frac{Wn}{Tgn}$$

where Tg is the theoretical glass transition temperature in kelvins for the polymer; W1, W2, W3, . . . , and Wn being the fractions by weight of each component of the copolymer; and Tg1, Tg2, Tg3, . . . , Tgn being the theoretical glass transition temperatures in kelvins of the homopolymer having an average molecular weight of at least 20,000, as obtained from the individual monomer units of the polymer [reference: T. G. Fox, Bul. Am. Phys. Soc., No. 3, page 123 (1956)] [reference: Polymer Handbook 3rd edition, 1989, published by J. Wiley & Sons, J. Brandrup and E. H. Immergut].

A more experimental technique for measuring the glass transition temperature is differential scanning calorimetry (DSC) which measures the variation in the enthalpy of a polymer with temperature. In particular, it is possible to use a DSC 30 type calorimeter from the supplier Mettler.

The glass transition temperature (Tg) can be measured in application of the ASTM D3418-97 standard, by DSC analysis of enthalpy over a temperature range of −100° C. to +150° C. with heating at a rate of 10° C./min in aluminum crucibles. The sample containing the polymer in the dry state or in solution in a solvent is placed in a crucible. Once the polymer is in solution, the solvent is initially allowed to evaporate for 24 h at ambient temperature and at 50% relative humidity.

The glass transition temperature (Tg) can also be measured by dynamic and mechanical temperature analysis (DMTA).

In order to measure the glass transition temperature (Tg) of the polymer, viscoelasticity meter tests are performed using a DMTA type apparatus from TA Instruments (model DMA2980) on a sample of polymer film having a thickness of about 200±50 μm, a width of 10 mm, and a length of 15 mm, after drying for 24 h at 23° C. and under relative humidity of 50%-55%. The sample is subjected to traction. The sample is subjected to a static force of 0.01 N having superposed thereon sinusoidal displacement of ±8 μm at a frequency of 1 hertz (Hz). Action thus takes place in the linear range with low levels of deformation. This traction is applied to the sample at temperatures lying in the range −150° C. to +220° C., with temperature varying at a rate of 3° C. per minute.

The complex modulus E*=E′+1E″ of the polymer under test is then measured as a function of temperature.

From these measurements, the conservation dynamic modulus E′ and the loss modulus E″ are deduced together with the damping power: tan δ=E″/E′.

Thereafter, the curve of values for tan δ as a function of temperature is plotted. This curve presents at least one peak. The glass transition temperature Tg of the polymer corresponds to the temperature at which the tip of the peak is located. It is generally about 15° C. greater than the theoretical Tg.

When the curve presents at least 2 peaks (in which case the polymer presents at least 2 Tgs) then the value taken for the Tg of the polymer under test is the temperature at which the curve presents the peak of greatest amplitude (i.e. corresponding to the greatest value for tan δ; under such circumstances, the “majority” Tg on its own is considered as being the Tg value for the polymer under test).

Preferably, the glass transition temperature of sequenced polymers suitable for use in foundations of the invention is measured by DSC.

Unless specified otherwise, the Tg values given for the first and second sequences in the present description are theoretical Tg values.

This formation of a specific morphology can be controlled in obvious manner by the nature of the blocks, and also by the molecular weight and the length of the blocks.

The sequence of variable conformation, advantageously having a Tg value that is less than or equal to 30° C., or even 20° C., is a homopolymer or a copolymer and is preferably derived in full or in part from one or more monomers which are such that the homopolymers prepared therefrom have glass transition temperatures that are less than or equal to 30° C., or even 20° C.

The polydispersity index of the sequenced polymer is preferably greater than 2, preferably greater than or equal to 2.5, and better greater than or equal to 2.8, and in particular lies in the range 2.8 to 6.

The polydispersity index I of the polymer is equal to the ratio of its weight average mass Mw over its number average mass Mn. Mw and Mn are determined as specified above.

The weight average mass (Mw) of the polymer of the invention is preferably less than or equal to 150,000 grams per mol (g/mol) and lies for example in the range 35,000 g/mol to 150,000 g/mol, and better in the range 45,000 g/mol to 100,000 g/mol.

The number average mass (Mn) of the polymer of the invention is preferably less than or equal to 40,000, and lies for example in the range 10,000 to 40,000, and better in the range 12,000 to 25,000.

Advantageously, the sequenced polymer is a linear polymer.

The sequenced polymer is preferably soluble and/or dispersible at ambient temperature (25° C.) at a concentration of active material of at least 10% by weight in at least one solvent, in particular an organic solvent.

Each sequence of a block of the sequenced polymer is derived from one type of monomer or from a plurality of different types of monomers.

This means that each sequence may be constituted by a homopolymer or a copolymer; the copolymer constituting the sequence may in turn be alternating or random.

The intermediate segment, if any, comprising at least one component monomer of the first sequence of at least one component monomer of the second sequence of the polymer is generally itself a random polymer.

The sequenced polymer may be obtained by radical polymerization or by polymerization in solution using the method described in documents EP 1 411 069 and WO 04/028488, the contents of which are incorporated herein by reference.

As mentioned above, the sequence of variable conformation and the sequence of limited conformation have different glass transition temperatures.

The difference between the glass transition temperatures of the sequences of limited and variable conformation is generally greater than 20° C., preferably greater than 30° C., and better greater than 40° C.

### Sequence of Variable Conformation

As mentioned above, the sequence or block of variable conformation possesses a glass transition temperature of less than 30° C., in particular lying in the range −100° C. to +25° C., e.g. in the range −90° C. to +20° C., and better in the range −80° C. to 0° C.

This variable conformation sequence, which advantageously has Tg less than or equal to 30° C. or better less than or equal to 20° C. is a homopolymer or a copolymer and is preferably obtained in full or in part from one or more monomers, which are such that the homopolymers prepared from said monomers have glass transition temperatures less than or equal to 20° C.

In particular, the monomers whose homopolymers have Tg values less than or equal to 20° C. and from which the sequences having Tg values less than or equal to 20° C. in the polymer of the invention is/are preferably derived, are themselves preferably selected from the following monomers:
acrylates having the formula CH₂═CHCOOR₃, where R₃ represents a linear or branched C₁ to C₁₂ alkyl group, preferably other than the teriobutyl group, in which there may optionally be interposed one or more heteroatoms selected from O, N, S, said alkyl group possibly also being substituted by one or more substituents selected from hydroxyl groups and halogen atoms (Cl, Br, I, and F), or else R₃ represents a C₁ to C₁₂ polyoxyethylene (POE) alkyl with an oxyethylene motif repeated 5 to 30 times, e.g. methyoxy-POE, or else R₃ represents a polyoxyethylene group having 5 to 30 ethylene oxide motifs;
methacrylates having the formula CH₂═C(CH₃)—COOR₄, where R₄ represents a linear or branched C₄ to C₁₂ alkyl group optionally including one or more interposed heteroatoms selected from O, N, and S, said alkyl group possibly also being substituted by one or more substituents selected from hydroxyl groups and halogen atoms (Cl, Br, I, F);
vinyl esters of formula R₅—Co—O—CH═CH₂, where R₅ represents a linear or branched C₄ to C₁₂ alkyl group;
C₄ to C₁₂ vinyl and alkyl ethers, such as vinyl and methyl ether and vinyl and ethyl ether;
C₄ to C₁₂ N-alkyl acrylamides, such as N-octylacrylamide; and
copolymers thereof.

Particularly preferred monomers for the sequence of variable conformation are alkyl acrylates in which the alkyl chain comprises 1 to 10 or 1 to 4 carbon atoms, such as methyl acrylate, isobutyl acrylate, ethyl-2-hexyl acrylate, and copolymers thereof.

The proportion of the variable conformation sequence in which Tg is generally less than 30° C., or less than 20° C., lies in the range 5% to 75%, preferably in the range 15% to 50%, and better in the range 25% to 45% by weight relative to the weight of the sequenced polymer.

### Limited-Conformation Sequence

The sequence or block of limited conformation advantageously has Tg greater than or equal to 40° C., e.g. Tg lying in the range 40° C. to 120° C., preferably greater tan 50° C., better greater than 60° C.

This limited-conformation sequence is a homopolymer or a copolymer and is preferably derived completely or in part from one or more monomers, which are such that the homopolymers prepared from said monomers have glass transition temperatures greater than or equal to 40° C.

More preferably, this first sequence is a homopolymer, constituted by a single type of monomer (for which the Tg of the corresponding homopolymer is greater than or equal to 40° C.).

The monomers having homopolymers with glass transition temperatures greater than or equal to 40° C. and from which the limited conformations sequences of the sequenced polymer are preferably derived are themselves preferably selected from the following monomers:
methyacrylates having the formula CH₂═C(CH₃)—COOR₁, in which R₁ represents a linear or branched alkyl group containing 2 to 4 carbon atoms, such as a methyl, ethyl, propyl, or isobutyl group, said alkyl group possibly also being substituted by one or more substituents selected from hydroxyl groups and halogen atoms (Cl, Br, I, F) or in which R₁ represents a C₄ to C₁₂ cycloalkyl group;
acrylates having the formula CH₂═CH—COOR₂, in which R₂ represents a C₄ to C₁₂ cycloalkyl group such as isobornyl acrylate or a terio butyl group;
(meth)acrylamides having the formula:

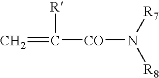

where R₇ and R₈ are identical or different, each representing a hydrogen atom or a linear or branched C₁ to C₁₂ alkyl group such as an n-butyl, t-butyl, isopropyl, isohexyl, isoocytyl, or isononyl group, and in which R₇ represents H, and R₁ represents a 1,1-dimethyl-3-oxobutyl group; and R′ designates H or a methyl group, example monomers that can be mentioned are N-butylacrylamide, N-t-butylacryamide, N-isopropylacrylamide, N,N-dimethylacrylamide, and N,N-dibutylacrylamide;
(meth)acrylic acid;
styrene and derivatives thereof such as chlorostyrene; and
copolymers thereof.

The monomers that are particularly preferred for the limited conformation sequence are methyl methacrylate, isobutyl methacrylate, isobornyl (meth)acrylate, trifluoroethyl methacrylate, styrene, (meth)acrylic acid, and copolymers thereof.

The proportion of the limited conformation sequence of Tg advantageously greater than or equal to 40° C. preferably lies in the range 20% to 90%, better in the range 30% to 80%, and better still in the range 50% to 70% by weight relative to the weight of the sequenced polymer.

In a particular embodiment, each of the variable conformation and limited conformation sequences may include a minority fraction of at least one consecutive monomer of the other sequence. Each of these sequences, in addition to the monomers mentioned above, may also contain one or more different monomers of nature and quantity that are preferably selected in such a manner that the sequence in which they are to be found has the desired glass transition temperature.

### Intermediate Segment

As mentioned above, the sequenced copolymer may contain an intermediate segment with Tg lying in the range 20° C. to 40° C. The sequenced polymer may also present at least one segment having Tg lying in the range 20° C. to 40° C., or in the range 30° C. to 40° C.

The intermediate segment which has Tg lying it the range 20° C. to 40° C. may be a homopolymer or a copolymer.

This segment having Tg lying in the range 20° C. to 40° C. may come in full or in part from one or more monomers, which are such that the homopolymer prepared from said monomers has a glass transition temperature lying in the range 20° C. to 40° C.

The intermediate segment having Tg lying in the range 20° C. to 40° C. may be derived in full or in part from monomers which are such that the corresponding homopolymer has a Tg value greater than or equal to 40° C. and monomers which are such that the corresponding homopolymer has a Tg value less than or equal to 20° C.

When this segment is a homopolymer, it is derived from one or more monomers, which are such that the homopolymers prepared from said monomers have glass transition temperatures lying in the range 20° C. to 40° C. The intermediate segment may be a homopolymer, constituted by a single type of monomer (with the corresponding homopolymer having a Tg lying in the range 20° C. to 40° C.).

The monomers for which the homopolymer has a glass transition temperature lying in the range 20° C. to 40° C. are preferably selected from n-butyl methacrylate, cyclodecyl acrylate, neopentyl acrylate, isodecylacrylamide, and mixtures thereof.

When the segment having a Tg lying in the range 20° C. to 40° C. is a copolymer, it is derived in full or in part from one or more main monomers, of nature and concentration that are selected in such a manner that the Tg of the resulting copolymer lies in the range 20° C. to 40° C.

Advantageously, the intermediate segment having Tg lying in the range 20° C. to 40° C. is a copolymer derived in full or in part:
from main monomers for which the corresponding homopolymer has Tg greater than or equal to 40° C., e.g. Tg lying in the range 40° C. to 150° C., preferably greater than or equal to 50° C., e.g. lying in the range 50° C. to 120° C., better greater than or equal to 60° C., e.g. lying in the range 60° C. to 120° C., as described above; and/or
from main monomers for which the corresponding homopolymer has Tg less than or equal to 20° C., e.g. Tg lying in the range −100° C. to 20° C., preferably less than or equal to 15° C., in particular lying in the range −80° C. to 15° C., and better less than or equal to 10° C., e.g. lying in the range −50° C. to 0° C., as described above, said monomers being selected in such a manner that the Tg of the copolymer forming the first sequence lies in the range 20° C. to 40° C.

Such main monomers are selected, for example, from methyl methacrylate, isobornyl methacrylate and acrylate, butyl acrylate, ethyl-2-hexyl acrylate, and mixtures thereof.

As a non-limiting illustration of sequenced copolymers suitable for the invention, mention can be made more particularly of the following copolymer variants:
a limited conformation sequence having Tg greater than or equal to 40° C., e.g. having Tg lying in the range 70° C. to 110° C., which is a methyl methacrylate and acrylic acid copolymer; and
a variable conformation sequence having Tg less than or equal to 20° C., e.g. lying in the range 0° C. to 20° C., which is a methyl acrylate homopolymer.

In a second variant, the copolymer of the invention may comprise:
a limited conformation sequence having Tg greater than or equal to 40° C., e.g. lying in the range 70° C. to 110° C., constituted by a methyl methacrylate and acrylic acid and trifluoroethyl methacrylate copolymer; and
a variable conformation sequence with Tg less than or equal to 20° C., e.g. lying in the range 0° C. to 20° C., which is a homopolymer of the methyl acrylate

In a third variant, the copolymer of the invention may comprise:
a limited conformation sequence with Tg greater than or equal to 40° C., e.g. lying in the range 85° C. to 115° C., constituted by a copolymer of isobornyl acrylate, and isobutyl methacrylate; and
a variable conformation sequence of Tg less than or equal to 20° C., e.g. lying in the range −85° C. to −55° C., constituted by a homopolymer of the ethyl-2-hexyl acrylate.

In a fourth variant, the copolymer of the invention may comprise:
a limited conformation sequence of Tg greater than or equal to 40° C., e.g. lying in the range 95° C. to 125° C., which is a copolymer of isobornyl acrylate and isobornyl methacrylate; and
a variable conformation sequence of Tg less than or equal to 20° C., e.g. lying in the range −35° C. to −5° C., which is an isobutyl acrylate homopolymer.

The following are most particularly suitable for the invention:
copolymers in which the limited conformation sequence comprises isobornyl acrylate and isobutyl methacrylate at respective concentrations of 40% and 30% by weight of the copolymer and in which the variable conformation sequence comprises ethyl-2-hexyl acrylate at a concentration of 30% by weight of the copolymer; and
copolymers in which the limited conformation sequence comprises isobornyl acrylate and isobornyl methacrylate, each at a concentration of 35% by weight relative to the weight of the copolymer, and in which the variable conformation sequence comprises isobutyl acrylate at a concentration of 30% by weight relative to the weight of the copolymer.

Advantageously, the first and second sequences of the sequenced polymer are incompatible with each other.

Such polymers are described for example in documents EP 1 411 069 or WO 04/028488 which are incorporated herein by reference.

### Active Ingredients

The foundation may comprise at least one cosmetically or dermatologically active ingredient. Cosmetically, dermatologically, hygienically or pharmaceutically active ingredients which may be used in the compositions of the invention which may be mentioned are moisturizers (polyol such as glycerin), vitamins, (C, A, E, F, B or PP), essential fatty acids, essential oils, ceramids, sphingolipids, liposoluble sunscreens or sunscreens in the form of nanoparticles, specific active ingredients for the treatment of skin (protective agents, antibacterials, anti-wrinkle agents, etc), self-tanning agents. Said active ingredients may, for example, be used in concentrations of 0 to 20% and especially 0.001% to 15% with respect to the total composition weight.

The foundation may also contain ingredients which are routinely used in cosmetics, such as thickeners, surfactants, oligo elements, moisturizers, softeners, sequestrating agents, fragrances, alkalizing or acidifying agents, preservatives, anti-oxidants, UV screens, colorants, or mixtures thereof.

Depending on the envisaged type of application, the foundation may comprise constituents conventionally used in the fields under consideration which are present in a quantity appropriate for the desired form.

### Packaging

The foundation may be presented in various forms, in the form of an oily or aqueous solution, an oily or aqueous gel, an oil-in-water emulsion, a water-in-oil emulsion, a multiple emulsion, a dispersion of oil-in-water by means of vesicles situated at the oil/water interface.

The foundation may be proposed to the user on its own or in association with at least one magnetic device enabling it to be subjected after application to a magnetic field in order to obtain a local variation in lightness.

By way of example, FIG. 2 shows a kit 18 comprising a compact 19 containing the foundation P, capable of closing in leaktight manner when not in use, and a magnetic device constituted in the example shown by a permanent magnet 20. The compact 19 may also house an applicator 21 such as a brush enabling the foundation to be applied.

Where appropriate, the magnetic device 20 may comprise at least one electromagnet and a source of electrical energy, e.g. together with a switch enabling the user to power the electromagnet selectively.

In a variant that is not shown, the foundation is contained in a receptacle suitable for being sprayed on the skin, e.g. by means of an airbrush or any other spray device. In use, after the foundation has been applied, the user can modify the orientation of the magnetic bodies contained herein by exposing them to magnetic field lines produced by the magnetic device.

The exposure to the magnetic field may, for example, comprise a first pass of the magnet for conferring a common orientation on the magnetic bodies relative to the skin, followed by a second pass restricted to regions in which it is desired to modify lightness.

In a variant shown in FIG. 3, the foundation is present on a backing 22. The foundation can be transferred to the skin when the backing 22 is pressed thereagainst.

### PROPOSED EXAMPLES

The following compositions are examples of foundation in which lightness and hue angle can be varied under the effect of a magnetic field. The percentages are expressed by weight.

### Example I

| | |
|---|---|
| Magnesium sulfate | 1.5 |
| Sodium carboxymethyl cellulose | 0.5 |
| Distearyldimethylammonium-modified hectorite | 1 |
| Cyclopenta dimethylsiloxane | 16 |
| Glycerol | 5 |
| Mixture of oxyethylenated | 9 |
| polymethylcetyldimethylmethysiloxane, isostearate | |
| polyglycerol (4 moles), hexyllaurate | |
| Water | 31.6 |
| A mixture of acetyl ethylene stearate, glyceryl | 0.3 |
| tri-stearate | |
| Composite pigment * | 25 |
| Poly dimethylsiloxane (viscosity: 5 centistokes | 6 |
| cst)) | |
| 1,2-pentanediol | 3 |

* Composite pigment comprising a magnetic core coated in a mixture comprising brown iron oxide (CI: 77491) 18%, yellow iron oxide (CI: 77492) 19%, black iron oxide (CI: 77499) 5%, and titanium oxide (anatase) (CI: 77891) 58%.

### Example II

| | |
|---|---|
| Magnesium sulfate | 1.5 |
| Sodium carboxymethyl cellulose | 0.5 |
| Distearyldimethylammonium-modified hectorite | 1 |
| Cyclopenta dimethylsiloxane | 16 |
| Glycerol | 5 |
| Mixture of oxyethylenated | 9 |
| polymethylcetyldimethylmethysiloxane, isostearate | |
| polyglycerol (4 moles), hexyllaurate | |
| Water | 31.6 |
| A mixture of acetyl ethylene stearate, glyceryl | 0.3 |
| tri-stearate | |
| Composite pigment ** | 25 |
| Poly dimethylsiloxane (viscosity: 5 cst) | 6 |
| 1,2-pentanediol | 3 |

** Composite pigment comprising a magnetic core coated in a mixture made up of titanium oxide (anatase) (CI: 77891) 45%, black iron oxide (CI: 77499) 5%, yellow iron oxide (CI: 77492) 19%, brown iron oxide (CI: 77491) 27%, and ultramarine blue (CI: 77007) 2%.

### Example III

| | |
|---|---|
| Magnesium sulfate | 1.5 |
| Sodium carboxymethyl cellulose | 0.5 |
| Distearyldimethylammonium-modified hectorite | 1 |
| Cyclopenta dimethylsiloxane | 16 |
| Glycerol | 5 |
| Mixture of oxyethylenated | 9 |
| polymethylcetyldimethylmethysiloxane, isostearate | |
| polyglycerol (4 moles), hexyllaurate | |
| Water | 31.6 |
| A mixture of acetyl ethylene stearate, glyceryl | 0.3 |
| tri-stearate | |
| Composite pigment comprising a magnetic core | 1.58 |
| coated in brown iron oxide | |
| Composite pigment comprising a magnetic core | 18.17 |
| coated in titanium oxide (anatase) | |
| Composite pigment comprising a magnetic core | 4.56 |
| coated in yellow iron oxide | |
| Composite pigment comprising black iron oxide | 0.69 |
| Poly dimethylsiloxane (viscosity: 5 cst) | 6 |
| 1,2-pentanediol | 3 |

In all these examples, after the foundation has been applied, the user can use a magnetic device to lighten or darken the foundation depending on the orientation of the field lines relative to the skin.

Field lines parallel to the skin tend to orient the magnetic particle parallel thereto, thus tending to increase the area of each particle that is exposed to incident light radiation. When the field lines are oriented substantially perpendicularly to the skin, the magnetic particles tend to become oriented perpendicularly thereto, thereby reducing the area exposed to incident light. This can be used to vary lightness.

The term “comprising a” should be understood to be synonymous with “comprising at least one” unless specified to the contrary, and “lying in the range” should be understood as including the end values.

1. A foundation including at least one coloring agent having non-zero magnetic susceptibility, the foundation in bulk presenting a hue angle h lying in the range 30° to 70° and lightness L* lying in the range 25 to 80, the coloring agent being selected in such a manner that the foundation, under the action of a magnetic field, can present maximum variation in hue angle Δhₘₐₓ that is less than or equal to 5°, and variation in lightness ΔL* that is greater than or equal to 4.

2. A foundation according to claim 1, wherein the foundation is a liquid foundation.

3. A foundation including:
at least one aspherical composite pigment comprising a core having non-zero magnetic susceptibility and coated at least in part by a mixture of coloring materials selected so that the foundation presents in bulk lightness L* lying in the range 25 to 80 and a hue angle h lying in the range 30° to 70°.

4. A foundation according to claim 3, wherein the foundation is a liquid foundation.

5. A foundation including:
a mixture of at least two aspherical composite pigments each comprising a core having non-zero magnetic susceptibility and coated at least in part in at least one coloring material, the coloring materials of the mixture being selected so that the foundation in bulk presents lightness L* lying in the range 25 to 80, and a hue angle h lying in the range 30° to 70°.

6. A foundation according to claim 5, wherein the foundation is a liquid foundation.

7. A foundation according to claim 1, in which the hue angle h of the foundation lies in the range 35° to 65°.

8. A foundation according to claim 6, in which the hue angle h of the foundation lies in the range 35° to 55°.

9. A foundation according to claim 1, in which the hue angle h of the foundation lies in the range 40° to 60°.

10. A foundation according to claim 1, in which the hue angle h lies in the range 46° to 50° and the lightness L* lies in the range 55 to 65.

11. A foundation according to claim 1, in which the hue angle h lies in the range 49° to 54° and the lightness L* lies in the range 55 to 65.

12. A foundation according to claim 1, in which the hue angle h lies in the range 52° to 54° and the lightness L* lies in the range 60 to 65.

13. A foundation according to claim 1, in which the hue angle h lies in the range 50° to 55° and the lightness L* lies in the range 40 to 55.

14. A foundation according to claim 1, in which the variation in lightness ΔL* is greater than or equal to 5.

15. A foundation according to claim 1, in which the variation in lightness ΔL* is greater than or equal to 6.

16. A foundation according to claim 1, in which the coloring agent comprises at least one composite pigment, comprising a magnetic core of aspherical shape coated at least in part by a shell.

17. A foundation according to claim 16, in which the composite pigment is arranged to produce a color at least in part by an absorption phenomenon.

18. A foundation according to claim 16, in which the shell comprises a mixture of coloring materials.

19. A foundation according to claim 1, in which the coloring agent comprises a plurality of magnetic pigments of different colors.

20. A foundation according to claim 19, in which the coloring agent comprises a plurality of magnetic composite pigments of different colors, each comprising a magnetic core of elongate space.

21. A foundation according to claim 1, having viscosity lying in the range 6.5 Poise to 38 Poise.

22. A foundation according to the claim 21, having viscosity lying in the range 11.5 Poise to 19 Poise.

23. A foundation according to claim 1, including at least one organic solvent.

24. A foundation according to claim 23, wherein the solvent is a volatile organic solvent.

25. A foundation according to claim 24, including at least one oil.

26. A foundation according to claim 25, wherein the oil is a volatile oil.

27. A foundation according to claim 1, including at least one film-forming agent.

28. A foundation according to claim 1, in which the coloring agent presenting non-zero magnetic susceptibility comprises metallic iron.

29. A foundation according to claim 28, wherein the coloring agent comprises soft iron.

30. A foundation according to claim 1, including a sequenced polymer.

31. A method of making up the skin, the method comprising:
applying a foundation as defined in any preceding claim to the skin; and
subjecting the foundation as applied in this way to a magnetic field in such a manner as to modify the orientation of at least some of the particles of the coloring agent(s) presenting non-zero magnetic susceptibility.

32. A method according to claim 31, wherein the foundation is applied to the face.

33. A method according to claim 31, wherein the modification to the orientation of the coloring agent(s) presenting magnetic susceptibility is such as to obtain variation in the lightness ΔL* of the foundation as applied in this way that is greater than or equal to 4.

34. A method according to claim 31, wherein makeup of the light/dark type is achieved.

35. A kit for implementing the method as defined in claim 31, the kit comprising:
a receptacle or a backing carrying a foundation as including at least one coloring agent having non-zero magnetic susceptibility, the foundation in bulk presenting a hue angle h lying in the range 30° to 70° and lightness L* lying in the range 25 to 80, the coloring agent being selected in such a manner that the foundation, under the action of a magnetic field, can present maximum variation in hue angle Δhₘₐₓ that is less than or equal to 5°, and variation in lightness ΔL* that is greater than or equal to 4; and
at least one magnetic device enabling a magnetic field to be generated.
